(19) United States
(12) Patent Application Publication
(10) Pub. No.: US 2019/0083221 A1
(43) Pub. Date: Mar. 21, 2019

(54) MALE INCONTINENCE WRAP

(71) Applicant: Jamnee Jugmahun, Brooklyn, NY (US)

(72) Inventor: Jamnee Jugmahun, Brooklyn, NY (US)

(21) Appl. No.: 15/709,809

(22) Filed: Sep. 20, 2017

(51) Int. Cl. A61F 2/00

(52) U.S. Cl. CPC A61F 2/0009

(57) ABSTRACT

A sealable wrap for placement over a penis that includes: a padded section where the padded section is between a first fold and a second fold; a first section on the first fold adjacent to the padded section; a second section on the second fold adjacent to the padded section; a horizontal fold below the first section, second section and padded section; three adjoining sections below the horizontal fold corresponding to the first section, second section and padded section; and adhesive tape to secure the sealable wrap in a closed position. The adhesive tape may be attached to an outside edge of the first section. The three adjoin sections below the horizontal fold may include a third section below the first section, a fourth section below the padded section, where the fourth section is padded and a fifth section below the third section.

### BACKGROUND OF THE INVENTION

### Field of Invention

The present invention relates to a wrap for application on a male appendage.

### Description of Related Art

Many individuals are inflicted with incontinence which specifically relates to the accidental or involuntary loss of urine from the bladder. Many times incontinence is caused by other health problems or due to medication that the individual may be taking. Incontinence normally requires the individual to have frequent urinations during the day and unfortunately many times uncontrollable urinations. This condition inevitably requires the individual to wear disposable underwear to absorb urine and to avoid embarrassing soiling of their clothing. Further, many bedbound patients must use disposable underwear to control urination. Because of the use of the disposable underwear, individuals must frequently change these underwear over the course of a day to avoid skin irritations. As a result, it would be advantageous to have the specific padding for a male's penis. In particular, such padding could be used to absorb urine immediately upon exiting from the penis to avoid excessive soiling of the disposable underwear. Such padding could essentially prolong the life of the disposable underwear and then allow for easier clean up of the individual.

### SUMMARY OF THE INVENTION

The present invention relates to a sealable wrap for placement over a penis that includes: a padded section where the padded section is between a first fold and a second fold; a first section on the first fold adjacent to the padded section; a second section on the second fold adjacent to the padded section; a horizontal fold below the first section, second section and padded section; three adjoining sections below the horizontal fold corresponding to the first section, second section and padded section; and adhesive tape to secure the sealable wrap in a closed position. The adhesive tape may be attached to an outside edge of the first section. The three adjoin sections below the horizontal fold may include a third section below the first section, a fourth section below the padded section, where the fourth section is padded and a fifth section below the third section. The fourth section includes adhesive tape on the outside edge. Preferably, the sealable wrap includes a fold sequence to convert the wrap from an open position to a closed position.

### BRIEF DESCRIPTION OF DRAWINGS

FIG. 1 depicts an expanded view of a wrap in accordance with the present invention.

FIG. 2 depicts the wrap according to the present invention folded.

### DETAILED DESCRIPTION

The present invention relates to an incontinence wrap, where a padded section is placed at the center of the wrap. The padded section is surrounded by a first section and a second section where a first vertical fold adjoins the padded section in the first section and a second vertical fold joins the padded section in the second section. These three sections lie above three additional sections below a horizontal fold. The lower sections of the wrap include a third section, a fourth section and a fifth section. The fourth section abuts the padded section and the wrap folds at the horizontal fold to secure the wrap in a closed position. This wrap is adapted for a placement for a penis to receive urine released there from.

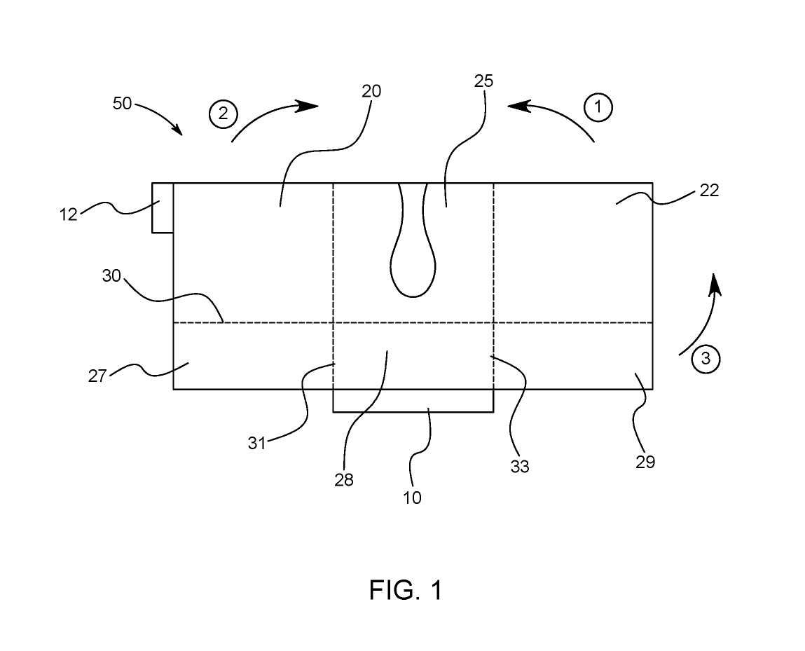

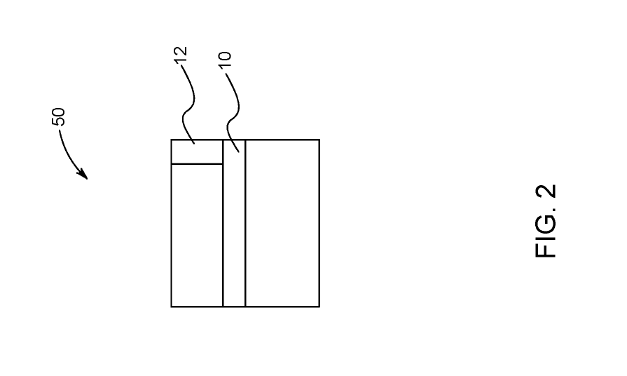

FIG. 1 shows the expanded view of a wrap 50 in accordance with the present invention. The wrap 50 includes a padded section 25 that is adapted to receive a male's penis thereupon. This padded section 25 joins a first section 20 which is separated by a first fold 31. It also is joined to a second section 22, which is separated from the padded section 25 by a second fold 33. These three sections are joined and fold consistently with arrows shown in FIG. 1. Below the second section 22 is a horizontal fold 30 that extends across all three sections. The horizontal fold 30 enables the complete wrapping of the wrap 50 to seal the opening a user's penis. Below the horizontal fold 30, a third section 27, a fourth section 28 which is also padded and a fifth section 29 are shown. These sections fold upward to seal the wrap in a closed position as noted by the arrow 3. Adhesive tape is provided at two sections of the wrap 50. Adhesive tape 12 is shown at the outer edge of the first section 20 and adhesive tape 10 is shown at the outer edge of the fourth section 28. In a closed position as shown in FIG. 2, wrap 50 closes where the tape seals the sections in a closed position. As shown section 22 folds over to the top of padded section 25 and then section 20 goes over section 22 then with a final fold at the horizontal fold 30 where the sections 27 and 29 have adjoined with section 28 as a result of the first two folds and then the portion below fold 30 folds upward to seal the wrap as shown in FIG. 2.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A sealable wrap for placement over a penis comprising:
a. a padded section where the padded section is between a first fold and a second fold;
b. a first section on the first fold adjacent to the padded section;
c. a second section on the second fold adjacent to the padded section;
d. a horizontal fold below the first section, second section and padded section;
e. three adjoining sections below the horizontal fold corresponding to the first section, second section and padded section; and
f. adhesive tape to secure the sealable wrap in a closed position.

2. The sealable wrap according to claim 1, where the adhesive tape is attached to an outside edge of the first section.

3. The sealable wrap according to claim 1, where the three adjoin sections include a third section below the first section, a fourth section below the padded section, where the fourth section is padded, and a fifth section below the third section.

4. The sealable wrap according to claim 3, where the fourth section include adhesive tape on the outside edge.

5. The sealable wrap according to claim 1, further including a fold sequence to convert the wrap from an open position to a closed position.
